(19) United States
(12) Patent Application Publication
(10) Pub. No.: US 2002/0087167 A1
(43) Pub. Date: Jul. 4, 2002

(54) Skin exfoliator

(76) Inventor: Kathleen M. Winitsky, Delray Beach, FL (US)

Correspondence Address: Gregory A. Nelson Akerman Senterfitt, Fourth Floor, 222 Lakeview Avenue, P.O. Box 3188, West Palm Beach, FL, 33402-3188, US

(21) Appl. No.: 09/752,872

(22) Filed: Dec. 29, 2000

(51) Int. Cl. A61B 17/50

(52) U.S. Cl. 606/131

(57) ABSTRACT

A skin exfoliator is provided for exfoliating the surface of a user's skin. In one arrangement, the skin exfoliator can have an abrasive sheet, an absorbent pad affixed to the abrasive sheet and a moisturizer disposed in the absorbent pad. In addition, an adhesive can be disposed between the abrasive sheet and the absorbent pad thereby affixing the abrasive sheet to the absorbent pad. In another arrangement, the abrasive sheet can have a base having a top and bottom surface. Moreover, abrasive particulates can be dispersed on the top surface of the base and affixed to the base with an adhesive. The abrasive sheet can be an abrasive paper or, alternatively, an abrasive pad. In another variation, a moisturizer can be disposed in the absorbent pad. This moisturizer can be in liquid or powder form.

### BACKGROUND OF THE INVENTION

1. Technical Field

The invention relates generally to cosmetics, and more particularly to skin exfoliators for removing dead skin cells.

2. Description of the Related Art

Currently, millions of people use a wide variety of products and procedures to improve the appearance of their skin. For example, personal hygiene products such as alpha-hydroxy acids, RETIN-A® and RENOVA® manufactured by Ortho Dermatological in Puerto Rico, and facial scrubs enable a person to exfoliate their skin. This exfoliation process enables a person to remove dead skin cells. Although products such as those listed above are generally sufficient, more effective treatments are available. One such treatment is microdermabrasion. In microdermabrasion, micro-crystals are vacuumed through a hand piece and directed at an angle onto an area of the patient's skin. Using microdermabrasion, a skin-care technician can affect a superficial skin polishing.

Microdermabrasion treatments are known to be lengthy and expensive sessions. For example, a single microdermabrasion session typically can last thirty minutes. Moreover, microdermabrasion treatments can cost more than $90. Thus, what is needed is a simple, cost-effective solution for performing skin exfoliation.

### SUMMARY OF THE INVENTION

The present invention can be used in a simple, cost-effective solution for skin exfoliation. In one inventive arrangement, a skin exfoliator can include an abrasive sheet, an absorbent pad affixed to the abrasive sheet, and a moisturizer disposed in the absorbent pad. In another arrangement, the skin exfoliator can further include an adhesive disposed between the abrasive sheet and the absorbent pad. In this arrangement, the adhesive can affix the abrasive sheet to the absorbent pad. In yet another inventive arrangement, the moisturizer can be a liquid moisturizer. Alternatively, the moisturizer can be liquid activated. In yet another arrangement, the abrasive sheet can include a base having top and bottom surfaces and abrasive particulates dispersed on the top surface and affixed to the abrasive pad with the adhesive. For example, the abrasive sheet can be an abrasive paper. As another example, the abrasive sheet can be an abrasive pad.

### BRIEF DESCRIPTION OF THE DRAWINGS

There are presently shown in the drawing embodiments which are presently preferred, it being understood, however, that the invention is not limited to the precise arrangements and instrumentalities shown.

FIG. 1 is a perspective view illustrating a skin exfoliator in accordance with the inventive arrangements.

FIG. 2 is a side view of the skin exfoliator of FIG. 1 containing a breakable bladder.

FIG. 3 illustrates a side view of the skin exfoliator of FIG. 1.

FIG. 4 illustrates a skin exfoliator containing an absorbent pad.

FIG. 5 illustrates a skin exfoliator containing a non-absorbent pad.

### DETAILED DESCRIPTION OF THE INVENTION

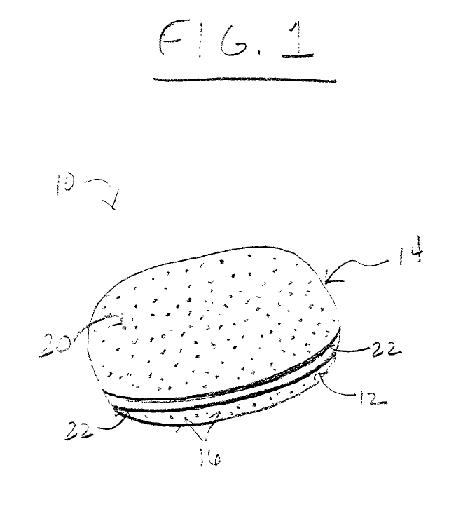

FIG. 1 illustrates a skin exfoliator 10 in accordance with the inventive arrangements. As shown in FIG. 1, the skin exfoliator 10 can include an absorbent pad 12 and an abrasive sheet 14. The abrasive sheet 14 can include an exfoliating surface and a non-exfoliating surface. In one arrangement, the absorbent pad 12 can be a non-allergenic sponge; however, the invention is not so limited as any other suitable absorbent material may be used. An adhesive 22 can be used to affix the absorbent pad 12 to the abrasive sheet 14. An adhesive 22 also can be used to affix abrasive particulates 20 to the abrasive sheet 14. In one arrangement, the adhesive 22 is a non-toxic, fast drying glue; however, the invention is not limited in either regard, as any adhesive may be used to secure the components of the invention. The absorbent pad 12 can contain a moisturizer 16. The moisturizer 16 can be dispersed throughout the absorbent pad 12.

Notably, the moisturizer 16 can provide relief to areas of skin that have been exfoliated by the skin exfoliator 10. In one embodiment, the moisturizer 16 can be a liquid moisturizer that has been previously absorbed by the absorbent pad 12 during the manufacturing process. Alternatively, the moisturizer 16 can be a liquid activated moisturizer. Under this arrangement, the moisturizer 16 contained in the pad is initially in powder form and can be hydrated by adding water or any other suitable liquid.

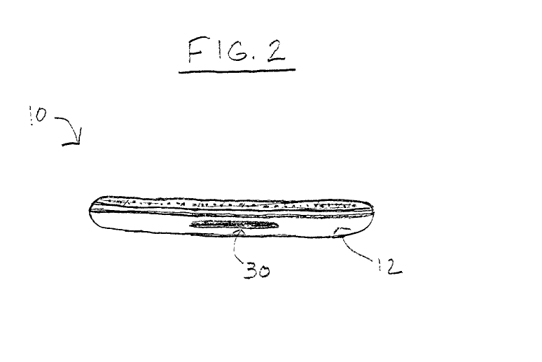

Notably, the moisturizer 16 can be contained in a breakable bladder disposed in the absorbent pad 12. FIG. 2 is a side view of an absorbent pad 12 which contains such a breakable bladder 30. The breakable bladder 30 can be constructed of a layer of plastic or any other non-porous material. Moreover, the breakable bladder 30 can be disposed at any location within the absorbent pad 12, including the surface of the absorbent pad 12 opposite the surface that is used during the exfoliating process.

In operation, the moisturizer 16 can be released into the absorbent pad 12 by squeezing, puncturing or otherwise breaching the seams of the bladder 30 so that the moisturizer 16 can be forced into the absorbent pad 12. Significantly, by enclosing the moisturizer 16 in the breakable bladder 30, the integrity of the skin exfoliator 10 can be preserved during its shipment and storage. In an alterative arrangement, the hydrating liquid used to mix with the powder to form the moisturizer, also, can be stored in a breakable bladder. As before, this arrangement can preserve the integrity of the moisturizing powder and hydrating liquid. In operation, the absorbent pad 12 can be squeezed which can cause the breakable bladder 30 containing the hydrating liquid to burst. As a result, the moisturizing powder and hydrating liquid can combine to form the moisturizer 16. In this arrangement, the user is not required to mix any ingredients, as is required when the liquid moisturizer 16 is initially in powder form.

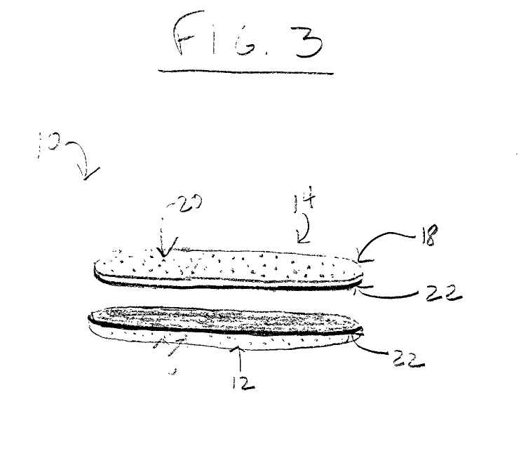

FIG. 3 illustrates a side view of the skin exfoliator 10. As shown in FIG. 3, the abrasive sheet 14 can include a base 18 having top and bottom surfaces and a plurality of abrasive particulates 20 dispersed on the top surface of the base 18. In one arrangement, the abrasive sheet 14 can be constructed of a paper of the type commonly used as a backing to conventional abrasive sheets. Alternatively, the abrasive sheet 14 can be constructed of a fabric, such as a linen or cotton cloth; however, the invention is not limited in this regard, as the abrasive sheet 14 can be constructed of other types of cloth. In another arrangement, the abrasive sheet 14 can be any suitable non-porous material such as rubber or neoprene. The abrasive particulates 20 preferably are fine sand particles that can be suitable for use in microdermabrasion. The abrasive particulates 20 can be randomly dispersed on the abrasive sheet 14; however, the invention is not so limited as the abrasive particulates 20 can be dispersed on the abrasive sheet 14 in an orderly fashion. In one arrangement, the abrasive particulates can be aluminum oxide, silicon carbide or any other similar abrasive material.

In operation, first abrasive sheet 14 can be applied to the skin surface of a human face and rubbed gently. The rubbing action can slightly abrade the skin resulting in the removal of dead skin cells. Subsequently, the absorbent pad 12 can be loaded with the moisturizer 16. In the case where a bladder holds either hydrating liquid or pre-mixed moisturizer, the bladder can be broken and the hydrating liquid or pre-mixed moisturizer forced into the absorbent pad 12. Once the absorbent pad 12 is loaded with the moisturizer 16, the moisturizer 16 can be applied to the exfoliated portion of the skin surface by gently rubbing the absorbent pad 12 on the area of skin that has been exfoliated. In consequence, the application of the moisturizer 16 can lubricate, hydrate and soothe the exfoliated portion of the skin surface. In addition, use of the moisturizer 16 can promote skin healing.

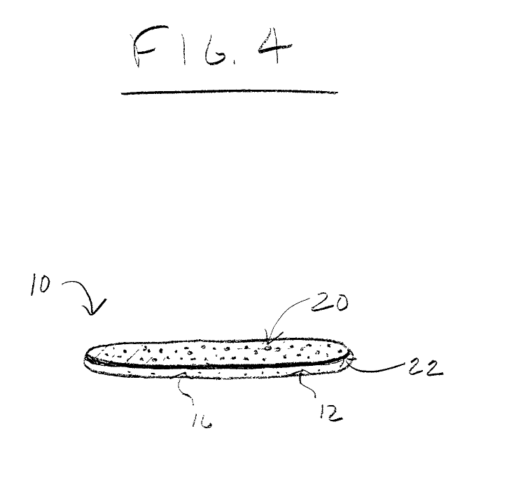

FIG. 4 illustrates an alternative arrangement of the skin exfoliator 10 in which the exfoliator 10 can include an absorbent pad 12, an adhesive layer 22 and a layer of abrasive particulates 20. In one arrangement, an adhesive 22 can be affixed to the top surface of the pad 12. Further, the abrasive particulates 20 can be dispersed on the adhesive 22. Under this arrangement, a moisturizer 16 can be dispersed in the absorbent pad 12. This moisturizer 16 can be a liquid moisturizer or can be a liquid activated moisturizer. The operation of the skin exfoliator 10 is similar to the previously discussed embodiments; however, the need for an abrasive sheet 14 has been eliminated.

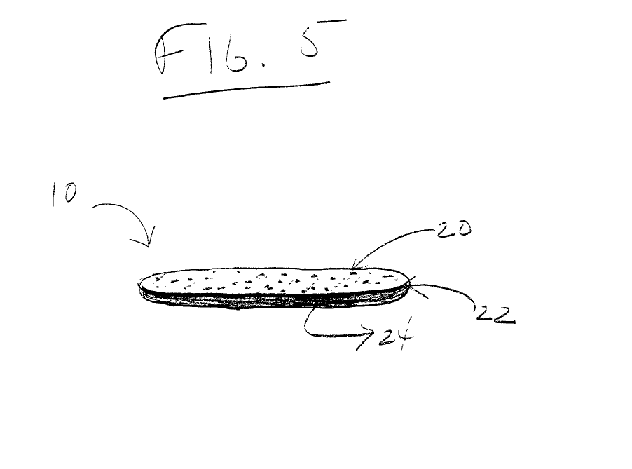

FIG. 5 illustrates an alternative arrangement of the skin exfoliator 10 discussed in FIG. 4. In this arrangement, the skin exfoliator can include a non-absorbent pad 24, an adhesive layer 22 and a layer of abrasive particulates 20. The non-absorbent pad 24 can be any suitable material capable of resisting liquid absorption. In one arrangement, an adhesive 22 can be affixed to the top surface of the pad 24. Further, the abrasive particulates 20 can be dispersed on the adhesive 22. The operation of the skin exfoliator 10 in this arrangement is similar to the previously discussed embodiments; however, the skin exfoliator 10 is more resistant to degradation and can be used a greater number of times.

I claim:

1. A skin exfoliator, comprising:
an abrasive sheet;
an absorbent pad affixed to said abrasive sheet; and,
a moisturizer disposed in said absorbent pad.

2. The skin exfoliator according to claim 1, wherein said abrasive sheet is an abrasive paper.

3. The skin exfoliator according to claim 2, wherein said abrasive paper comprises:
a base having top and bottom surfaces; and
abrasive particulates dispersed on said top surface and affixed to said abrasive paper with an adhesive.

4. The skin exfoliator according to claim 1, wherein said abrasive sheet is an abrasive pad.

5. The skin exfoliator according to claim 4, wherein said abrasive pad comprises:
a base having top and bottom surfaces; and
abrasive particulates dispersed on said top surface and affixed to said abrasive pad with an adhesive.

6. The skin exfoliator according to claim 1 further comprising an adhesive disposed between said abrasive sheet and said absorbent pad, said adhesive affixing said abrasive sheet to said absorbent pad.

7. The skin exfoliator according to claim 6, wherein said abrasive sheet is an abrasive paper.

8. The skin exfoliator according to claim 7, wherein said abrasive paper comprises:
a base having top and bottom surfaces; and
abrasive particulates dispersed on said top surface and affixed to said abrasive paper with an adhesive.

9. The skin exfoliator according to claim 6, wherein said abrasive sheet is an abrasive pad.

10. The skin exfoliator according to claim 9, wherein said abrasive pad comprises:
a base having top and bottom surfaces; and
abrasive particulates dispersed on said top surface and affixed to said abrasive pad with an adhesive.

11. The skin exfoliator according to claim 1, wherein said moisturizer is a liquid moisturizer.

12. The skin exfoliator according to claim 11, wherein said abrasive sheet is an abrasive paper.

13. The skin exfoliator according to claim 12, wherein said abrasive paper comprises:
a base having top and bottom surfaces; and
abrasive particulates dispersed on said top surface and affixed to said abrasive paper with an adhesive.

14. The skin exfoliator according to claim 11, wherein said abrasive sheet is an abrasive pad.

15. The skin exfoliator according to claim 14, wherein said abrasive pad comprises:
a base having top and bottom surfaces; and
abrasive particulates dispersed on said top surface and affixed to said abrasive pad with an adhesive.

16. The skin exfoliator according to claim 1, wherein said moisturizer is liquid activated.

17. The skin exfoliator according to claim 16, wherein said abrasive sheet is an abrasive paper.

18. The skin exfoliator according to claim 17, wherein said abrasive paper comprises:
a base having top and bottom surfaces; and
abrasive particulates dispersed on said top surface and affixed to said abrasive paper with an adhesive.

19. The skin exfoliator according to claim 16, wherein said abrasive sheet is an abrasive pad.

20. The skin exfoliator according to claim 19, wherein said abrasive pad comprises:
a base having top and bottom surfaces; and
abrasive particulates dispersed on said top surface and affixed to said abrasive pad with an adhesive.

21. The skin exfoliator according to claim 1, wherein said abrasive sheet comprises:
a base having top and bottom surfaces; and
abrasive particulates dispersed on said top surface of said base and affixed to said base with an adhesive.

22. The skin exfoliator according to claim 21, wherein said abrasive sheet is an abrasive paper.

23. The skin exfoliator according to claim 21, wherein said abrasive sheet is an abrasive pad.

24. A skin exfoliator, comprising:
a microdermabrasive sheet; and
an absorbent pad affixed to said microdermabrasive sheet.

25. The skin exfoliator according to claim 24, wherein said microdermabrasive sheet is a microdermabrasive paper.

26. The skin exfoliator according to claim 25, wherein said microdermabrasive paper comprises:
a base having top and bottom surfaces; and
abrasive particulates dispersed on said top surface and affixed to said microdermabrasive paper with an adhesive.

27. The skin exfoliator according to claim 24, wherein said microdermabrasive sheet is a microdermabrasive pad.

28. The skin exfoliator according to claim 27, wherein said microdermabrasive pad comprises:
a base having top and bottom surfaces; and
abrasive particulates dispersed on said top surface and affixed to said microdermabrasive paper with an adhesive.

29. A skin exfoliator, comprising:
an absorbent pad having a top surface;
an adhesive disposed on said top surface of said absorbent pad; and
abrasive particulates dispersed on said top surface and affixed to said absorbent pad with said adhesive.

30. The skin exfoliator according to claim 29, wherein a moisturizer is disposed in said absorbent pad.

31. The skin exfoliator according to claim 30, wherein said moisturizer is a liquid moisturizer.

32. The skin exfoliator according to claim 30, wherein said moisturizer is liquid activated.
